(19) United States
(12) Patent Application Publication
(10) Pub. No.: US 2016/0106598 A1
(43) Pub. Date: Apr. 21, 2016

(54) Cool Tips

(71) Applicant: John Janis, JR., Westville, IL (US)

(72) Inventor: John Janis, JR., Westville, IL (US)

(21) Appl. No.: 14/518,041

(22) Filed: Oct. 20, 2014

(51) Int. Cl. A61F 13/38

(52) U.S. Cl. CPC A61F 13/38

(57) ABSTRACT

This invention is directed to a system for cleaning ears. The cleaning of ears with a basic dry, cotton swab is a common way of attempting to remove the accumulation of wax or other foreign materials. The moistened “Cool Tips” swabs will be used as a daily ear cleaner, disinfectant and/or refresher proving to be significantly more effective and rewarding to the consumer.

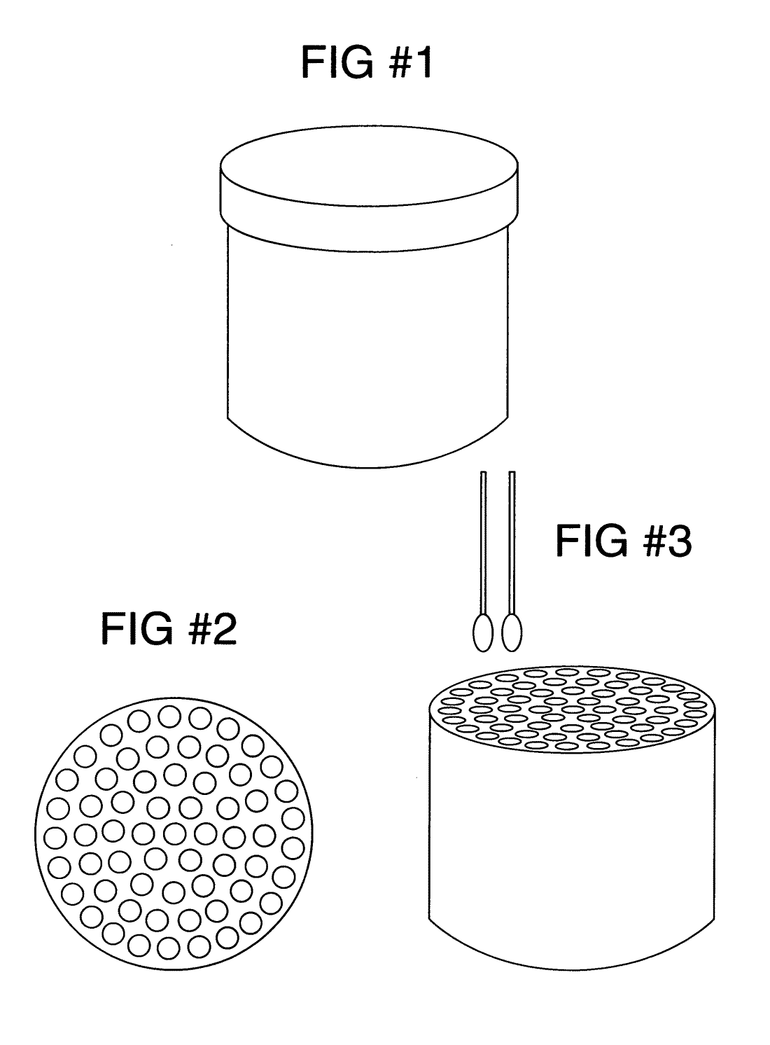

The common dry swab in and of itself does not do the job adequately, thus, not leaving the ear completely free and clean to accomplish its hearing function.

Providing a container of moist cotton swabs with an all-natural solution so that the ear can be cleaned confidently, with ease and thoroughness, avoids the cumbersome process of finding the desired solution, moistening the swab in an efficient manner, and using it without dripping or otherwise making a mess.

In accordance with my invention, “Cool Tips” offer a self-contained unit that enables one to clean their ears conveniently, easily, and effectively.

### DETAILED DESCRIPTION

“Cool Tips”. This ear cleaner assembly cleans and refreshes ears using a moist, not wet, single tip cotton swab. The cotton swab head is moistened with a solution of water, isopropyl alcohol, witch hazel, aloe, peppermint Oil.

This product is a unique self-containment of 90 swabs per jar. The swabs are upright and uniform inside that jar, held in place by a die cut sponge insert containing ninety 3 and 1/16 of an inch holes to hold swabs in place. Sponge insert is 2¼ inches in height and in a 3 inch tall jar. Only ¼ inch of the bottom of insert is wet, thus leaving the swab stem dry, and only the swab head moist.

The alcohol content allows the ear to dry quickly after using.

The peppermint oil content gives a wonderful cool and refreshing tingle, and a true clean fresh feeling after using this product. This product is a unique and novel way to care for ears. Consumers will know that their ears are clean and rid of any oil or sweat they may have accumulated that day or week gone by. Consumers will also enjoy the cool tingle they feel after using “Cool Tips”.

### DESCRIPTION OF DRAWINGS

FIG. #1 LABELED PLASTIC CONTAINER.

FIG. #2 TOP OF FOAM INSERT SHOWING EXACTLY NINETY HOLES PER INSERT.

FIG. #3 PLASTIC STEMMED SINGLE TIPPED COTTON SWABS DEMONSTRATING THE DOWNWARD POSITIONING OF EACH SWAB INTO THE FOAM INSERT.

FIG. #4 LABELED PLASTIC CONTAINER, INTERIOR VIEW OF PRODUCT INCLUDING DESIGN OF PACKAGING, PLASTIC LID FOR THE CONTAINER.

FIG. #5 PRODUCT ASSEMBLY COMPONENTS AND MATERIALS, DEMONSTRATION OF HOW EACH PLASTIC STEMMED, SINGLE TIPPED COTTON SWAB WILL BE INSERTED AND POSITIONED INTO THE FOAM INSERT FOR PACKAGING.

1. I, John Janis Jr. am bringing an invention of my own, to the market place to be sold as a health and beauty aid product. This product will be used as a daily ear cleaner, disinfectant and/or refresher. This product is intended for human use. This item will be considered a “unique” and “novel” cotton swab arrangement. Designed and created myself, with prototypes presently being tested. This product will be sold and marketed under the name “Cool Tips”. A small plastic container with screw on lid will contain a sponge insert, or type of absorbent material, moistened with a solution consisting of five ingredients, which are all over the counter items consisting of water, isopropyl alcohol, witch hazel, aloe, and peppermint oil. Further research may indicate changes to the final solution. The sponge insert in the plastic container will be drilled from top to bottom, stabilizing and organizing the cotton swab and the stem in a uniform upright position, design and style, keeping the swab head moist. The swab will be submerged from top to bottom down into the sponge. The stem will be plastic, and will not be affected by the liquid solution. The container will be approximately four (4) inches in diameter and three (3) inches deep with a safety seal and a screw on lid.
